(19) United States
(12) Patent Application Publication
(10) Pub. No.: US 2019/0043615 A1
(43) Pub. Date: Feb. 7, 2019

(54) PORTABLE EMERGENCY TELEHEALTH SYSTEM AND METHOD

(71) Applicants: Italo Ramachandra Subbarao, Hattiesburg, MS (US); Guy Paul Cooper, Avon, IN (US); Dennis Lott, Clinton, MS (US)

(72) Inventors: Italo Ramachandra Subbarao, Hattiesburg, MS (US); Guy Paul Cooper, Avon, IN (US); Dennis Lott, Clinton, MS (US)

(21) Appl. No.: 16/155,014

(22) Filed: Oct. 9, 2018

Related U.S. Application Data
(63) Continuation-in-part of application No. 15/668,927, filed on Aug. 4, 2017.
(60) Provisional application No. 62/569,038, filed on Oct. 6, 2017.
(60) Provisional application No. 62/371,333, filed on Aug. 5, 2016.

(51) Int. Cl. G16H 40/67; G16H 80/00; G16H 10/60; B64C 27/08; B64C 39/02

(52) U.S. Cl. CPC G16H 40/67; G16H 80/00; A61F 17/00; B64C 27/08; B64C 39/024; G16H 10/60

(57) ABSTRACT

A system and method for providing remote healthcare. The system includes a server and a medical kit. A remote patient in need of medical care or a bystander (user) may access and use the medical kit. The remote patient and/or other user may enter data of the condition of the patient via a user interface of the medical kit. A processor of the medical kit receives data and sends the data over a wireless network to the server via communications interfaces. The processor of the medical kit than receives data including instructions for using the plurality of medical tools from the server and produces the instructions to the user via the user interface.

### CROSS-REFERENCE TO RELATED APPLICATION

This application claims the benefit of priority of U.S. provisional application No. 62/569,038, filed Oct. 6, 2017 and is a continuation-in-part of U.S. non-provisional application Ser. No. 15/668,927, filed Aug. 4, 2017, which claims the benefit of priority of U.S. provisional application No. 62/371,333, filed Aug. 5, 2016, the contents of which are herein incorporated by reference.

### BACKGROUND OF THE INVENTION

The present invention relates to telehealth, and more particularly to a portable emergency telehealth system and method.

Currently, patients in need of care in remote, rural, and physically challenging areas (disaster areas, hazmat situations etc.) do not have access to care within critical periods of time. Ambulances and first responders have significant delays in responding to the above-mentioned patients. Further, there is currently no system or method for bystanders to adequately communicate with physicians and provide care under emergency situations to patients in need.

As can be seen, there is a need for a portable emergency telehealth system and method.

### SUMMARY OF THE INVENTION

In one aspect of the present invention, a system for providing remote healthcare comprises: an unmanned aerial vehicle comprising a wireless communications interface, and a hybrid powered engine, wherein the hybrid powered engine is powered by at least one battery and hydro carbon fuel; and a medical kit removeably coupled to the unmanned aerial vehicle and comprising a housing containing a plurality of medical tools.

These and other features, aspects and advantages of the present invention will become better understood with reference to the following drawings, description and claims.

### BRIEF DESCRIPTION OF THE DRAWINGS

FIG. 1 is a schematic view of an embodiment of the present invention;

FIG. 2 is a perspective view of an exemplary medical kit of the present invention;

FIG. 3 is a schematic view of an embodiment of the present invention used within a unmanned aerial vehicle;

FIG. 4 is a schematic view of an embodiment of the present invention interconnected with an emergency dispatcher;

FIG. 5 is a perspective view of a unmanned aerial vehicle of an embodiment of the present invention;

FIG. 6 is a bottom view of a unmanned aerial vehicle of an embodiment of the present invention;

FIG. 7 is a schematic side elevation view of a unmanned aerial vehicle of an embodiment of the present invention;

FIG. 8 is a schematic side elevation view of a unmanned aerial vehicle of an embodiment of the present invention; and

FIG. 9 is a block diagram of an embodiment of the present invention in use.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

The Healthcare Integrated Rescue Operations (HiRO) Scalable Emergency Telehealth System is a portable, scalable telehealth system intended to support medical emergencies, disasters, public health emergencies, remote medical emergencies, wilderness emergencies, CBRNE (Chemical, Biological, Radiological, Nuclear, Explosive) terrorist attacks, hazmat emergencies, multi casualty incidents, inaccessible crowd based multi-casualty events, and other health emergencies. The system provides a scalable emergency telehealth system platform that allows mobile/in field evaluation, triage, and treatment of multiple patients by lay person bystanders, and/or health providers that are responding to a disaster or other health emergency. The system is designed to empower lay person/bystanders or health providers through the use of telehealth technology to provide emergent medical care at the geographic location of need.

Features of the system may include an automated 911 or emergency response alert notification application or interface, a secure web-based telehealth portal with electronic health record and application interface, a cloud-based data management warehouse, a HiRO Telehealth Kit that utilizes lighting and haptic guidance that is accessed through the secure web-based telehealth portal (personal computer, tablet, smartphones, smart glasses including augmented reality interface), automated predictive analytical clinical monitoring and guidance medical application for triage and medical support for the physician and layperson bystander, modular container bins that can remotely unlock and open providing access for medications and other uses, and other wearable RFID/NFC patient triage and tracking devices.

The telehealth kits can be strategically accessed by lay person bystanders/heath providers through a variety of manners which include: Unmanned Aerial Vehicle (UAV), autonomous vehicle, emergency medical services vehicle (ambulance/police/fire engine), personal automobiles, and backpack access. Additionally, the kits can be mounted to the wall in a variety of settings including, but not limited to, malls, stadiums, amusement parks, resort hotels, national parks, and rural access health clinics.

Depending on the scale of the event, HiRO Telehealth System allows for telehealth system access through a secure web-based portal. The telehealth kit may be connected to the medical provider and dispatch system through the cloud or other wireless means. The medical provider accesses the patient virtually through a secure web based medical provider interface. The medical provider can access the space through multiple platforms (personal computer, tablet, smartphone, and smart glasses) including provider to patient (end to end) augmented reality interface.

The HiRO telehealth system is scalable and the platform automatically adjusts accordingly: individual provider treating an individual patient on a singular telehealth kit; individual provider treating multiple patients on a singular telehealth kit; multiple providers (primary physician and consultant(s)) treating an individual patient on a singular kit; individual provider treating multiple patients on multiple kits simultaneously; multiple providers (same or different location) treating patients from a singular event utilizing multiple kits; and multiple providers (same or different location) treating patients simultaneously from multiple events utilizing multiple kits.

The present invention may further utilize a smart phone application. Using the smart phone application, a user may provide notifications/alerts to the local 911 system, as well as provide secure telehealth conversations. The application may be used as a bridge until a telehealth kit arrives at scene or other first responders arrive at scene. Additionally, the smartphone application can utilize a unique hashtag alert.

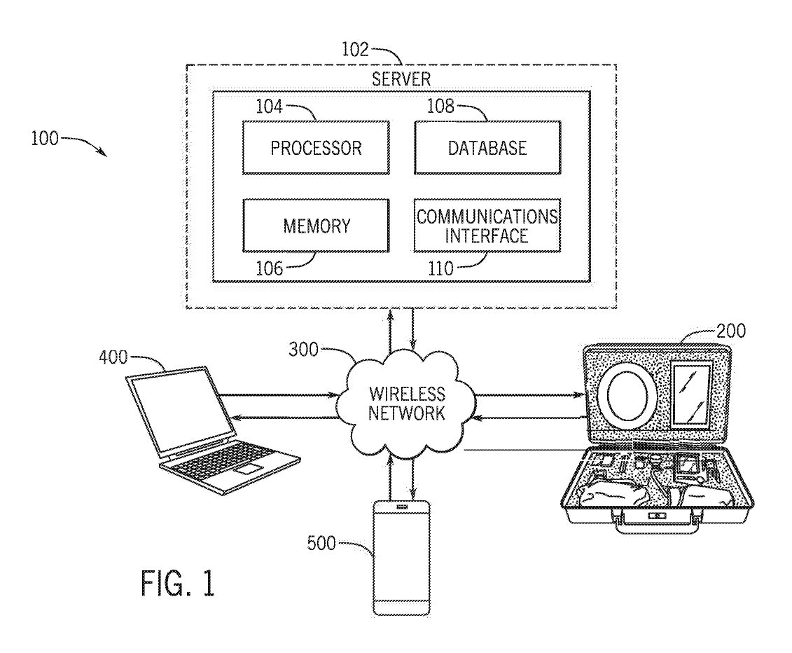

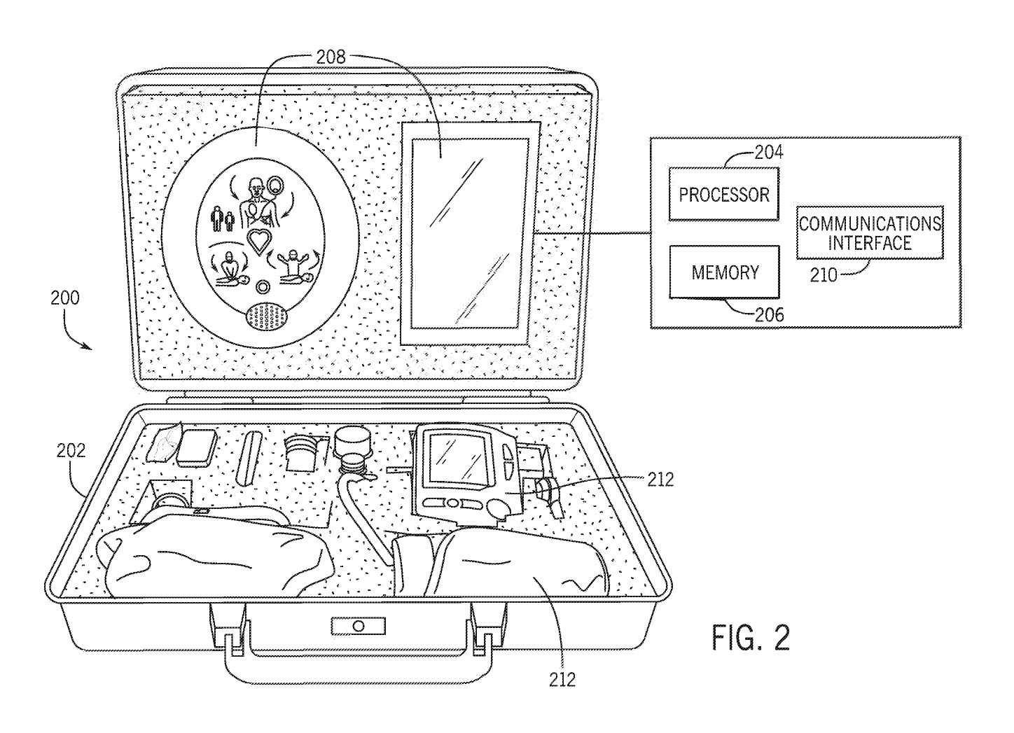

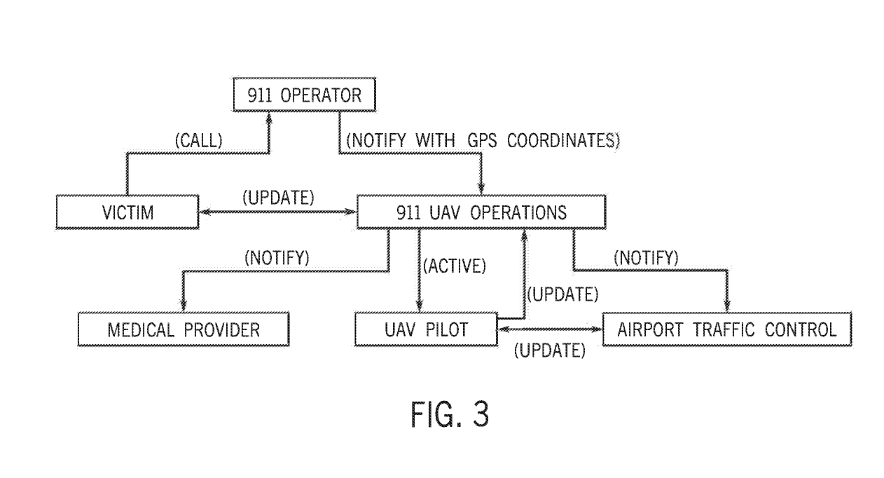

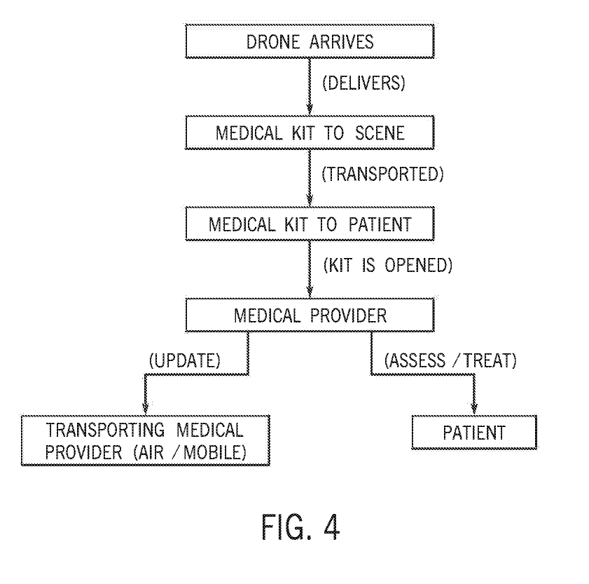

Referring to FIGS. 1 through 4, the present invention includes a system 100 for providing remote healthcare. The system 100 includes a server 102 having a processor 104, a memory 106, a database 108 and a communications interface 110. The system 100 further includes a medical kit 200 that is remote from the server 102. The medical kit 200 includes a housing 202 containing a processor 204, a memory 206, a user interface 208, a communications interface 210, and a plurality of medical tools 212. A remote patient in need of medical care or a bystander (user) may access and use the medical kit 200. The remote patient and/or other user may enter data of the condition of the patient via the user interface 208 of the medical kit 200. The processor of the medical kit 200 receives data and sends the data over a wireless network 300 to the server 102 via the communications interfaces 110, 210. The processor 204 of the medical kit 200 then receives data including instructions for using the plurality of medical tools from the server 102 and produces the instructions to the user via the user interface 208.

The wireless network 300 may refer to any interconnecting system capable of transmitting audio, video, signals, data, messages, or any combination of the preceding. The wireless network 300 may include all or a portion of a public switched telephone network (PSTN), a public or private data network, a local area network (LAN), a metropolitan area network (MAN), a wide area network (WAN), a local, regional, or global communication or computer network such as the Internet, an enterprise intranet, or any other suitable communication link, including combinations thereof.

The processors 104, 204 include hardware for executing instructions, such as those making up a computer program. The memories 106, 206 includes main memory for storing instructions such as computer program(s) for the processors 104, 204 to execute, or data for processors 104, 204 to operate on.

The database 108 includes mass storage for data or instructions such as the computer program. As an example, and not by way of limitation, the database 108 may include an HDD, a floppy disk drive, flash memory, an optical disc, a magneto-optical disc, magnetic tape, a Universal Serial Bus (USB) drive, a solid-state drive (SSD), or a combination of two or more of these. The database 108 may include removable or non-removable (or fixed) media, where appropriate. The database 108 may be internal or external to the server 102 where appropriate. In particular embodiments, the database 108 is non-volatile, solid-state memory.

The communication interfaces 110, 210 includes hardware, software, or both providing one or more interfaces for communication (e.g., packet-based communication) between the server 102, the medical kit 200 and the wireless network 300. As an example, and not by way of limitation, communication interfaces 110, 210 may include a network interface controller (NIC) or network adapter for communicating with a wireless NIC (WNIC) or wireless adapter for communicating with a wireless network, such as a WI-FI network. This disclosure contemplates any suitable network and any suitable communication interfaces 110, 210. As an example, and not by way of limitation, server 102 may communicate with an ad hoc network, a personal area network (PAN), a local area network (LAN), a wide area network (WAN), a metropolitan area network (MAN), or one or more portions of the Internet or a combination of two or more of these. One or more portions of one or more of the server 102 may be wired and/or wireless. As an example, the server 102 may communicate with a wireless PAN (WPAN) (e.g., a BLUETOOTH WPAN), a WI-FI network, a WI-MAX network, a cellular telephone network (e.g., a Global System for Mobile Communications (GSM) network), or other suitable wireless network or a combination of two or more of these. The server 102 may include any suitable communication interface 120 for any of these networks, where appropriate.

As mentioned above, the medical kit 200 includes a housing 202, a processor 204, a memory 206, a user interface 208, a communications interface 210 and medical tools 212. The medical kits 200 are portable telemedical kits that are designed for a variety of medical scenarios. Examples in which the kit could be deployed may include, but are not limited to: a) natural disaster: tornado, earthquake, hurricane, flooding; b) hazmat incident: organophosphate poisoning, cyanide poisoning; c) infectious disease epidemic: Ebola, cholera, measles; d) rural medical emergency: cardiac arrest, acute coronary syndrome, asthma, allergic reaction; e) wilderness medical emergency: hypothermia, dehydration, snake bite, environmental toxin; f) trauma emergency: multicar accident, mass shooting, extremity injury while hiking; g) bioterrorism; and h) military conflict or battlefield: penetrating chest and abdominal trauma, chemical exposure.

Medical kits 200 may vary by including different types of features and medical tools. Examples of types of medical kits may include, but are not limited to: a) general medical emergency kit: The kit can be used to evaluate individuals with these potential medical complaints such as cardiac arrest, chest pain, shortness of breath, dizziness, stroke or weakness, dehydration, allergic reaction, and unconscious patient; b) trauma medical emergency kit: The kit can be used for these potential trauma related medical complaints such as, but not limited to, head and spinal trauma, chest and abdominal trauma, extremity trauma, and lacerations; c) hazmat medical emergency kit: The kit can be used for these potential Hazmat related medical conditions such as, but not limited to, organophosphate poisoning, sarin nerve agent attack, cyanide poisoning or poisonous gas attack; d) infectious disease epidemic kit: The kit can be used for these potential infectious disease related medical conditions such as, but not limited, to Ebola, cholera, and measles; e) bioterrorism: The kit can be used bioterrorism related medical conditions such as, but not limited to, anthrax, Ebola, and small pox; and f) wilderness emergency medical kit: The kit can be used for the wilderness related medical conditions such as, but not limited to hypothermia, dehydration, high altitude sickness, allergic reaction, and environmental toxin.

The housing 202 of the medical kit 200 may include a strong case made of a polymer plastic or carbon fiber that is weatherproof, hard, and waterproof. The case may be aerodynamic allowing for reduced wind resistance and ease of transport. The housing 202 may be designed with a protective frame made of a polymer plastic, carbon fiber, ABS, nylon, or a similar material. The frame may be chemically resistant and provides support for internal modular compartments. The frame may be coupled to exterior sidewalls of the housing 202. The modular trays may contain the medical tools and may be made of polymer plastic, carbon fiber, ABS, Nylon, or similar materials.

The housing 202 may include the capability to be manually and remotely opened. The medical kit 200 can be opened with one hand and can include a hydraulic or spring-loaded capability to open. The mechanism can be unlocked either manually or automatically through a motor, solenoid, or other magnetic mechanism. Once the medical kit 200 is opened a light or contact based sensor may trigger the medical kit 200 to send an automated call or text to the medical dispatchers or emergency response system to contact a medical provider. Additionally, the sensor may turn on the medical kit user interface 208.

The housing 202 may be designed with a transport latch mechanism to allow for both delivery and pick-up on a multirotor UAV transport unmanned aerial vehicle. The medical kit 200 may include a transmitter to allow for tracking.

The medical kit 200 may include sirens, reflective tape, lighting, biometric sensors, monitors, shock absorbers, a parachute attachment, manual or remote-controlled wheels, hazmat sensors, antennas, tracking mechanisms, cameras, and/or locks. The medical kits 200 may further include an accelerometer, gyroscope, altimeter, global position system (GPS) tracking, and magnometer. The medical kit 200 may contain internal lighting to provide ambient lighting for the area. The modules may each include lighting, haptic sensors, climate control or a combination thereof which may be customizable to the needs of various medical uses. These modules can be an open-design or a closed-container bin design that are remotely unlocked and opened.

Electronic devices of the medical kit 200 may be powered by a battery. The battery may be charged through a variety of mechanisms such as: conduction, direct wire-to-outlet, and replacement of batteries.

The user interface 208 of the medical kit 200 may include, but is not limited to, a display, such as a touch screen, speakers, microphones, keyboards, mice, video cameras, smart glasses including augmented reality capabilities or a combination thereof. If the user interface includes a display, the display may be protected by an anti-microbial and chemically resistant film. The display may provide a live-stream of a medical provider and/or locally provided media. The devices of the medical kit 200 may connect together via wires or wireless means, such as BLUETOOTH and the like. The communications interface 208 may connect with the wireless network 300 via satellite, WiMax, cellular communications and other wireless means.

Additionally, the medical kit 200 may further include an emergency notification mechanical button that a user can press to alert the medical provider that the patient needs to be evaluated immediately. This is particularly valuable when a medical provider may be managing multiple patients with multiple kits, which is described in further detail below.

The medical tools 212 of the medical kit 200 may include diagnostic tools and treatment tools. The diagnostic tools may include, but are not limited to, temperature monitors, earpiece with microphone, heart rate monitors, blood pressure monitors, heart sounds monitors, lung sounds monitors, cardiac telemetry, pulse oximetry, blood glucose monitors, portable ultrasound monitor, and the like. The treatment tools may include, but are not limited to, an automatic external defibrillator, a tourniquet, bandages, gloves, CPAP, and gauze. The bins that may be remotely unlocked may include various medications, such as narcotics, inhalers, vaccines and antidotes, temperature control blood products, antibiotics, and the like.

The modular container bins are either lidded or un-lidded. Lidded container bins are secured in a closed position by locks and can be remotely unlocked and automatically opened by a medical provider over the server 102.

The modular container bins are designed so that a medical provider may unlock and give permission for the user to access the medication, diagnostic equipment, treatment interventions, and the like. If the container bin is remotely unlocked, a lighting may be activated to indicate what treatment to use. Furthermore, video may be streamed to a display located in the kit to demonstrate use of any required diagnostic equipment, treatment interventions, or medication administration.

In certain embodiments, the memory 206 of the medical kit 200 may include preloaded instructions that are produced based on user entered data. For example, if a medical kit 200 is accessed by a patient but there is a delay in reaching a medical provider, the medical kit 200 prompts the user to answer a few questions concerning the condition of the patient using the user interface 208. The processor 204 may match the entered data with one of the preloaded instructions and produce the instructions on the display. The instructions may provide immediate guidance and direction until the medical provider becomes available. The guidance can include directed action to the user with corresponding bins lighting up, as well as video streaming instructions on utilizations of particular applications. Additionally, the medical kit 200 may store data received and send the data to the server 102 once the connection has been established. An example may include the following: a patient is severely bleeding from there arm or leg, the user enters the data and the medical kit provides the instructions to grab the gloves and tourniquet and use the tools to properly stop the bleeding.

The medical kit 200 may further include a remote patient tracking and monitoring system. The medical kit 200 may store a unique wearable patient identifier such as a bracelet, pin, or sticker. The wearable identifier contains a unique RFID/NFC tracker. The wearable identifier can be scanned in through a sensor located in the medical kit 200. Once scanned, the appropriate EHR will flash open for the physician. The device may contain LED lighting and connect to the kit wirelessly. The light on the wearable identifier may change in conjunction with a digital triage tagging guidance, which is described in more detail below.

The system 100 of the present invention may be integrated with an emergency dispatcher system, such as a 911 operator. In such embodiments, an emergency dispatcher system is in communication with the server 102 when the medical kit 200 is in use. The emergency dispatcher system may be notified of the emergency based on the method in which the medical kit 200 is accessed and/or provided. For example, medical kits 200 that are designed for wall mounted access, such as at a mall, an amusement park, a resort hotel, a stadium and the like may notify an emergency dispatcher system when the medical kit is activated. In such embodiments, the medical kit 200 may include a sensor that triggers the medical kit 200 to send an automated call or text to an emergency dispatcher system. Alternatively, in a remote or wilderness scenarios such as a hiking accident in the woods or mountains, the hiker can use a wireless communications device, such as a smart phone, that notifies the emergency dispatcher system of the emergency which then triggers the server 102 to access a geolocation from the wireless communications device and potentially send an unmanned aerial vehicle caring the medical kit 200 to the location. Alternatively, social media can be utilized to alert the system 100 and emergency dispatcher system using active tagging of a specific hashtag.

In certain embodiments, the present invention may include a plurality of web-based communications portals produced by software loaded on the memory 106 of the server 102. The communications portals may be accessed by a plurality of computers remote from the server 102. The communications portals may facilitate communications between users of the remote computers and the user of the medical kit 200 over the Internet, telecommunications networks, or other wireless means mentioned above. The plurality of remote computers may include, but are not limited to a desktop, laptop, table smartphone, end to end augmented reality interface and the like. The communications portals may include, but are not limited to, a medical dispatch portal, a medical provider portal, an administrator portal, an observer portal and a consultant portal. Each of the plurality of communications portals may include permission-based access by the remote computers.

The medical dispatch portal may be accessed and used by a medical dispatcher. The medical dispatcher may enter salient information that is requisite for emergency response, which is sent to the server 102 and saved on the database 108. The information includes, but is not limited to, a time of notification, location of emergency, nature of emergency (type of medical problem), name of individual calling, and the like. The medical dispatcher may also select and notify a medical provider using the medical dispatch portal. If a medical kit needs to be sent to a remote location, the medical dispatcher may further enter instructions for an unmanned aerial vehicle (UAV) to deliver the medical kit to a remote area using the medical dispatch portal.

The medical provider portal may be accessed and used by the medical provider. The medical provider portal provides access to the entered information mentioned above and any additional health records of the patient. The medical provider portal may also include a billings page to record and collect future payments due. The medical provider portal allows the medical provider to connect and communicate to the user through the medical kit 200. The medical provider portal may further allow the medical provider to unlock bins and initiate lights to indicate which medical tools 212 are to be used.

In certain embodiments, the medical provider portal may be displayed in quadrants. A first quadrant may display the salient medical information on the patient so that the medical provider may enter a medical triage designation. The salient medical information is designed in tabular format and allows for documentation for each field. Fields may include, but are not limited to, chief compliant, history of present illness, past medical history, allergies, medications, review of systems, assessment, and plan. Once a medical triage designation is indicated (dead, critical, urgent, non-urgent) a corresponding color tab may be indicated with the patient name. Universal colors for triage include, black for dead, red for critical, yellow for urgent, and green for non-urgent.

A second quadrant may allow the medical provider to control portions of the medical kit 200 to provide remote guidance. The medical provider may select a certain treatment, diagnostics, or laboratory and the corresponding area in the medical kit 200 may light up, or haptic sensors of the bin may be engaged to guide the user. A corresponding video may be streamed to demonstrate how to apply or use the medication, treatment intervention, or diagnostic equipment. The remote guidance may also allow a medical provider to unlock and automatically open container bins that may contain medications or other products. The guidance activities may be automatically logged by date and time into the database 108 of the server 102.

A third quadrant may facilitate communication between the medical provider and the user. The third quadrant may include a secure visual and audio interface. The visual interface may be utilized through the display of the medical kit 200, the eyes of the bystander via visual technology, or through a smart phone application. A video camera embedded either within the medical kit 200 or through smart glasses with an augmented reality interface may allow the medical provider to view through the bystander's eyes.

A fourth quadrant may include a diagnostic data stream. The diagnostic data stream may include a real-time diagnostic streaming and tracking. The diagnostic streaming data that may be tracked and trended includes, but is not limited to: temperature, heart rate, blood pressure, pulse oximetry, blood glucose levels, cardiac electrode telemetry, and the like. In certain embodiments, medical provider portal visually alerts the medical provider if a vital sign is above or below a certain threshold. The notification may be an alert, such as a sound or light, or may include changing the color of the font in the patient's EHR.

The above-mentioned quadrants may be projected on a screen of the remote computer or via augmented reality. For further enhancement, an augmented reality visual may stream the vital signs and digital triage tagging guidance of the patient in the field. Triage color display and vital signs may also stream in the video display of the medical kit 200 and on any wearable device provided to the patient. Multiple patients and communications with consultants can be managed in the medical provider portal.

Once the encounter is completed, the medical provider can end the encounter and a billing page may be generated that may require some additional input. The billing page provides a log of the medical care provided and also includes critical information needed to bill for reimbursement. The medical provider portal may allow the physician to obtain a screen shot of the individual's driver's license or other identification ID, as well as a screen shot of the individual's insurance card. Other information that may be collected includes, but is not limited to, the patient's name, date of birth, personal address, phone number, and the like.

The administrator portal may be accessed by the emergency dispatchers. The administrator portal may provide the updated status of all activities. The updated status may include, but it not limited to, overall logs of patient encounters, medical kit use, medical provider participation, and billing information.

The observer portal may be used by other medical staff and professionals. The additional medical staff and professionals may monitor the patient remotely without permissions to modify treatment (this tier is used for roles such as first responders on the way to the call or medical personal waiting for the patient at a hospital). The observer portal is a designated observer page which views the medical provider page and actions that are being taken.

The consultant portal allows for an additional medical professional to monitor or assist in the treatment of the patient. The consultant portal allows the additional medical professional to communicate directly with the patient, bystander, or original medical provider over the wireless network 300. The additional medical professionals can be a specialist such as a neurologist providing guidance or other medical emergency specialist. The consultant port may also be used for support during infectious disease outbreak and epidemic management, as well as other large-scale disasters requiring trauma support.

The server 102 of the present invention may utilizes an automated predictive analytics software loaded on the memory 106 for clinical and triage support. The database 108 allows for real time data collection while the software instructs the processor 104 to monitor, and suggest action based on the scale of the event. The servers 102 predictive analytics software for triage and clinical support is used for both the medical provider and for the user in the field.

After the medical provider designates the patient's triage status on the patient's electronic health record, the server 102 virtually monitors the status by tracking and trending respective parameters (vital signs, etc.) and may change the triage depending on patient status. The triage designation can automatically be changed due to the predictive analytic software that is loaded on the memory 106 of the server 102. The software may raise the triage level based on vitals of the patient being recorded and sent to the server 102. Wearable devices provided to the patient may also contains a light that change in conjunction with the updated triage designation. Further, the medical provider may be notified if a vital sign or triage changes via the medical provider portal. For example, if the systolic blood pressure is less than 100, this will automatically cause the blood pressure font to turn red in the electronic health record within the medical provider portal. If a change does occur the system may automatically flash the patients tab, change the color tab accordingly and notify the medical provider. This is particularly useful if multiple kits are utilized for a mass casualty scenario.

As mentioned above, the medical kits 200 may be used for different purposes. The below example is of a medical kit 200 being used in conjunction with laboratory testing. In such embodiments, the medical kit 200 is designed and configured for use in unmanned aerial vehicle mobile laboratory use. The unmanned aerial laboratory system can transport the medical kits bi-directionally (to and from the facility) for use in various situations: natural disasters where roads may be inaccessible, and blood products are required for medical care (e.g. earthquake); remote locations such as third world countries where roads may be inaccessible; and rural health care facilities that may have prolonged delays in transporting specimens. The medical kits 200 may include a standard telemedical visual interface, diagnostics (e.g. Pulse Ox, BP, Temp), and specifically include: visual guidance to provide instruction to community health care worker; personal protective equipment; IV tubing, needles, alcohol swabs, and bandages; sample collection kits and tubes, analysis, transportation of blood, blood products (platelets, FFP, Cryoprecipitate etc.), bodily fluids (urine, stool, semen, etc.), and biopsy specimen samples; climate control compartments for cooling and heating; visual, lighting, and haptic guidance; and vaccine carrying capacity and compartment.

In such embodiments, the medical provider may log into the medical provider portal and generate a unique patient encounter. The medical provider can then obtain a brief history and physical on the patient and order the requisite labs. Certain symptoms (fever and bloody cough, fever and bloody diarrhea) may prompt the server to guide the medical provider to ask additional questions and to take some objective measurements (Temp, BP, HR), particularly with regards to infectious disease epidemic and outbreak management. Patients lab samples that are pooled regionally may be continuously scanned (predictive analytics) both for symptom and laboratory confirmation with regards to infectious disease and outbreak detection so that mitigation and response and can begin promptly.

The kits are designed such that the specimen collection tubes and compartments have an RFID chip for patient ID scanning and tracking via a RFID scan reader that is connected to the medical kit 200. The sample is scanned by the reader and is linked with the name of the patient and other variables (time, location, etc.) that are populated into the database 108. Additionally, QR codes may be utilized to track samples. Bluetooth® capabilities on the medical kit 200 allow for downloading patient information. The health care worker/user can also record other factors as necessary with the patient. GPS data may be consistently monitored regardless of how the sample data is tracked.

Specimen kits are tracked with varying user profiles: Maintenance (time since last cleaning, temperature/gyroscope/accelerometer readings, known spills since last cleaning, total samples loaded in case, flagged samples pre or post carrying); Transport team (patient data unknown. RFID or NFC list will be available, temperature/accelerometer/gyroscope data); Lab tech (QR code to RFID/NFC list for verification); and administrative (all of the above).

In certain embodiments, any positive laboratory sample or finding may be flagged and sent to the medical provider through the medical provider portal for them to review. If a laboratory sample is abnormal or positive for an infectious disease agent, the specimen tracking system may auto-alert the account of the health care worker/user to address the issue. Predictive analytics may be monitoring epidemic risk.

The following include an example of the characteristics of a lab medical kit 200. The medical kit 200 may be may to take the following measurements: internal environment sensors record temperature, pressure, magnetic fields, acceleration, gyroscopic data, tilt, and humidity; accelerometer and gyroscope data is recorded when they exceeded preconfigured thresholds; and infrared sensors measures the number of vials in the kit. The medical kit 200 design may include the following: a bottom tray may be made of lightweight chemically resistant plastics to include (abs, nylon, and varied other polymers); a bottom tray may be removable for cleaning or disposal; an RFID shielding may be present; samples may be kept in place through rubberized grommets and a security tray that comes between the disposable tray and the tube stopper; heavy duty fabric (kevlar or similar resistive fabric) line the kit to reduce fluid leakage if the case is punctured; and the tray locking system uses fixed kit points to lock the sample try into the kit. The data collecting device and power supply may be stored on outside of device and connected to the system through a waterproof adapter. Power may include solar charging, induction charging, rechargeable or disposable batteries made from lithium ion, lithium polymer, nickel cadmium or batteries with similar properties. Tamper evident seals may be placed on the case, tray, and samples. Biometric fingerprint sensors may be used in the processing of samples, unlocking of the kit, or retrieval of kit data. Samples may use an interlocking cap system that when forcibly unlocked it may make all samples unusable. If unauthorized system access is attempted multiple times the data may be deleted by the system. Fluid exposure materials may be placed throughout the kit. A cooling system may include a fan, Peltier or other electric cooling, wet or dry ice storing area, removable plastic cooling packs, radiators or heat sinks, thermal conducting gels or sealants to improve thermal conduction.

The below example is of a medical kit 200 being used in conjunction with poison control and response. The medical kit 200 may be designed to address emergency, poisonous ingestions or exposure. These emergencies include, but are not limited to: ingestions, overdoses, toxic stings or bites, envenomations, environmental encounters (poison ivy), industrial, occupational, or environmental accidents, organophosphate poisoning, cyanide poisoning, nerve agent exposure, bee stings, rattlesnake envenomations, beta blocker overdose, insulin overdose, as well as intentional chemical warfare (eg. nerve agents, cyanide), radiation, or burns. In such embodiments, the system 100 may include a poison control portal. A user or bystanders can trigger the system by selecting a clinical encounter button for poison ingestions and exposures. By pressing the button, automated actions may be instituted by the interface to include the opening of one or more locked access medication boxes, which allows for access to antidotes and other necessary medications. A video may guide the bystander to take actions to place diagnostic equipment on the patient as well.

The system auto-notifies poison control and allows telemedical access to a medical specialist. The specialist may have additional functionality on his/her interface to include specialized camera control. A specialized camera with higher resolution and infrared sensors may be included in these specific medical kits 200 to look at pupils, in addition to infrared sensors to take temperature and heart rate. The analytic software may help with identification of toxicological syndromes (Anticholinergic, Sympathomimetic, etc.) to assist with treatment. The interface may also allow the physician to the ability to remotely guide the user to access antidotes, and other specialized items.

For ingestions, the medical provider can take digital images with the camera for digital scanning and identification. The bottle and pills may be scanned against existing medication databases to identify the ingested substance. Snakes, spiders, plants, and other environmental toxins can also be pictured and scanned into the system. The visual file queries a database of existing toxins and medications providing a list of possible medications to the physician accompanied by relevant reference materials.

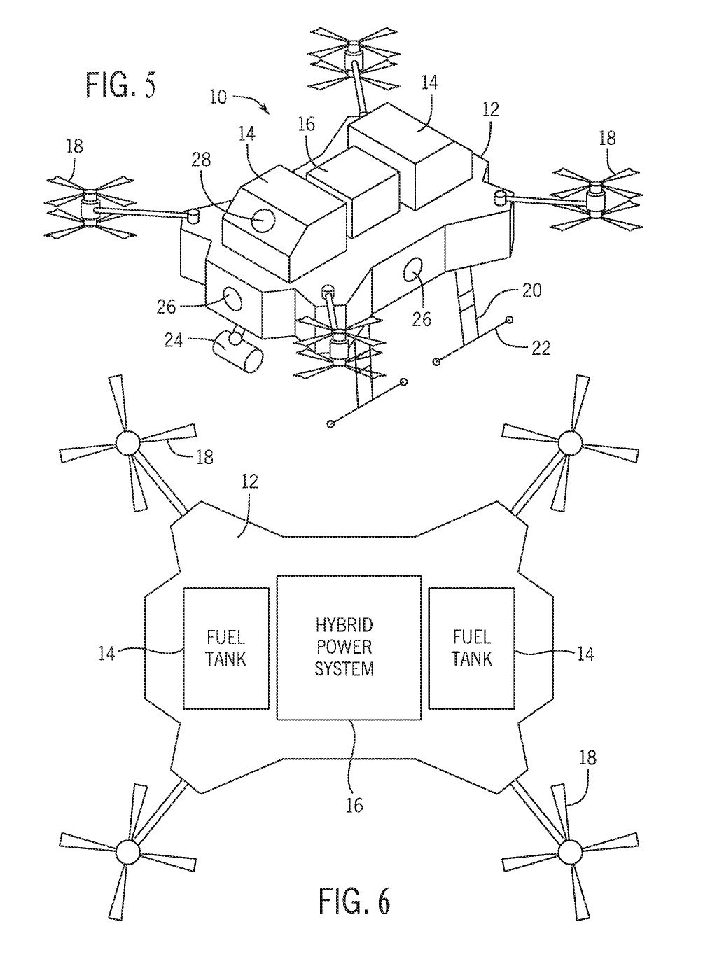

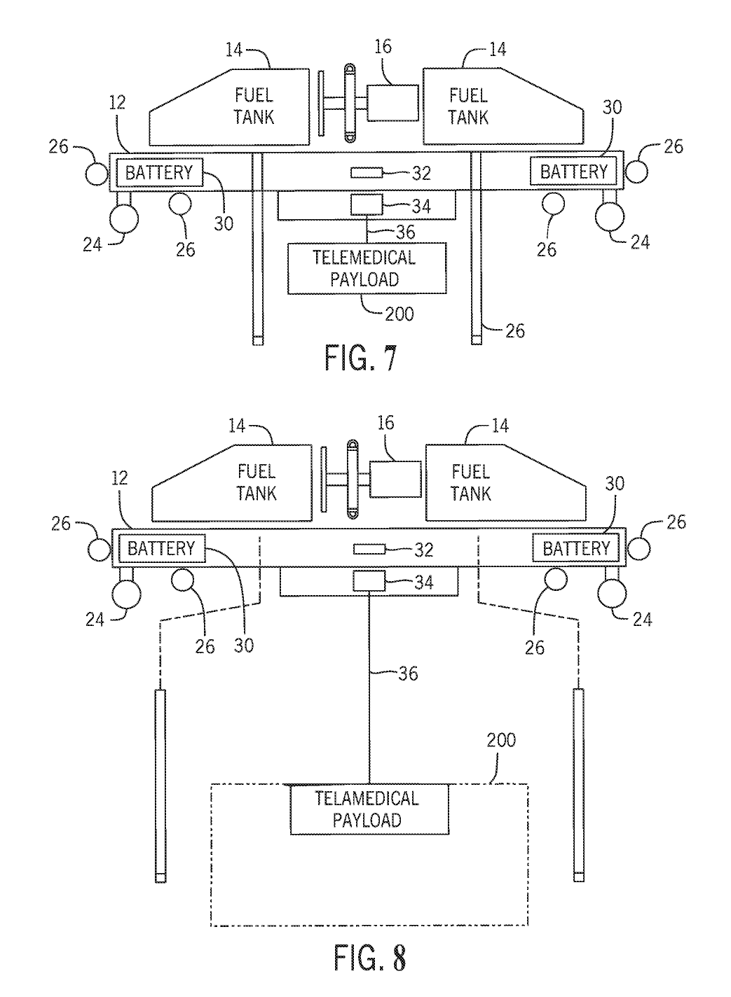

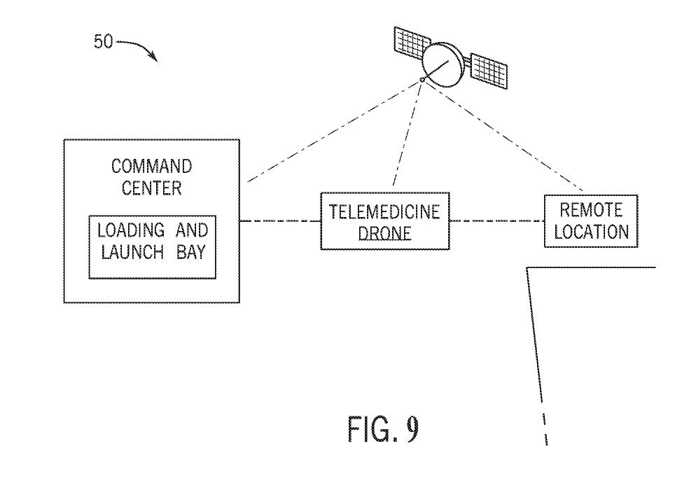

Referring to FIGS. 5 through 9, a hybrid transport unmanned aerial vehicle (UAV) 10 may be used to transport the medical kits 200. The hybrid transport UAV 10 utilizes hydro carbon fuel and battery power to increase the flight time with heavier payloads. The hybrid transport UAV 10 is built with several internal and external components that can provide a power source, carry medical kits 200 and/or other equipment or freight, and may include additional detachable components.

The UAV 10 includes a body 12. An engine 16 is coupled to the body 12. Propellers 18, such as four dual propellers 18, are coupled to the body 12 and are rotated by the engine 16. As mentioned above, the engine 16 is powered by batteries 30 and fuel. Fuel tanks 14 are coupled to the body 12. The fuel tanks 14 store and provide hydro carbon fuels to the engine 16. The batteries 30 are electrically coupled to the engine 16 and provide electricity to the engine 16.

The UAV 10 may further include additional components. For example, speakers 28, lights 26 and cameras 24 may be coupled to the body 12 of the UAV 10. The UAV 10 further includes an on-board computer 32 having a communications interface with wireless communication 50 capabilities. This allows users to remotely control the UAV 10 as well as communicate through the speakers 28 and utilize the lights 26 and cameras 24 to view the UAV's 10 point of view. In certain embodiments, the UAV 10 further includes a landing skid 20. Splints 22 are part of and removeably coupled to the landing skid 20. Therefore, in an emergency situation, the splints 22 may be removed from the landing skid 20 and used to aid a survivor in need.

The UAV 10 may further include a winch 34. The winch is coupled to the body 12 and electrically connected to the on-board computer 32 so that a remote user may control the winch 34. The winch 34 includes a cable 36 wrapped around a remotely controlled spool. A hook may be coupled to the end of the cable 36. The medical kit 200 may be releasably attached to the hook. In such embodiments, the UAV 10 may be flown to remote locations and the medical kit 200 may be lowered to survivors using the winch 34. Once the medical kit 200 has been delivered, the UAV 10 may return to a home base.

The hybrid engine 16 allows for ability to transport medical kits 200 to areas that are hard to reach due to distance and/or barriers. The UAV 10 can be used to transport a multitude of items to geographically difficult locations as the hybrid system gives the UAV 10 longer flight times, even with heavy payloads. The batteries 30 may also be used as an external power source to power needed equipment, whether medical or non-medical. For example, the UAV 10 may include outlets that electrically connect with the batteries 30. Additionally, the UAV 10 may include communications boosters as a relay system to help boost communication in areas with little service. In certain embodiments, the UAV 10 may also include an inner storage compartment that can be climate controlled. Blood or other products that need climate control may be transported within the UAV 10.

It should be understood, of course, that the foregoing relates to exemplary embodiments of the invention and that modifications may be made without departing from the spirit and scope of the invention as set forth in the following claims.

What is claimed is:

1. A system for providing remote healthcare comprising:
an unmanned aerial vehicle comprising a wireless communications interface, and a hybrid powered engine, wherein the hybrid powered engine is powered by at least one battery and hydro carbon fuel; and
a medical kit removeably coupled to the unmanned aerial vehicle and comprising a housing containing a plurality of medical tools.

2. The system of claim 1, further comprising a server comprising a computer having a processor, a memory, and a communications interface.

3. The system of claim 2, wherein the medical kit further comprises a processor, a memory, a user interface, and a communications interface, wherein the processor of the medical kit
receives data from a user via the user interface comprising a condition of a patient;
sends the data over a wireless network to the server via the communications interfaces;
receives data over the wireless network via the communication interface comprising instructions for using the plurality of medical tools based on the data comprising the condition of the patient; and
produces the instructions to the user via the user interface.

4. The system of claim 3, wherein the user interface of the medical kit comprises at least a display, a speaker and a microphone.

5. The system of claim 4, wherein the instructions are in the form live streaming video.

6. The system of claim 5, wherein the display is a touchscreen.

7. The system of claim 4, wherein the plurality of medical tools comprises a plurality of diagnostic tools and a plurality of treatment tools.

8. The system of claim 7, wherein the plurality of diagnostic tools comprises a temperature monitor, a heart rate monitor, a blood pressure monitor, a heart sound monitor, a lung sounds monitor, a cardiac telemetry, a pulse oximetry, a blood glucose monitor, or a combination thereof.

9. The system of claim 8, wherein a plurality of diagnostics taken by the diagnostic tools are sent to the server over the wireless network.

10. The system of claim 4, wherein the plurality of treatment tools comprises an automatic external defibrillator, a tourniquet, bandages, gloves, or a combination thereof.

11. The system of claim 3, wherein the medical kit further comprising a plurality of locked bins each comprising a medication.

12. The system of claim 11, wherein the plurality of locked bins are unlocked using the server.

13. The system of claim 1, wherein the unmanned aerial vehicle comprises a body comprising the battery, a fuel tank, the engine, and a landing skid coupled to the body, wherein splints are part of and removeably coupled to the landing skid.

14. The system of claim 13, wherein the unmanned aerial vehicle further comprises at least one speaker coupled to the body.

15. The system of claim 13, wherein the unmanned aerial vehicle further comprises at least one light coupled to the body.

16. The system of claim 13, wherein the unmanned aerial vehicle further comprises at least one camera coupled to the body.

17. The system of claim 13, wherein the unmanned aerial vehicle further comprising a winch coupled to the body and a cable extending from the winch, wherein the medical kit is removeable attached to the cable.

18. The system of claim 1, wherein the unmanned aerial vehicle comprises four dual propellers.
